(19) United States
(12) Patent Application Publication
(10) Pub. No.: US 2014/0100659 A1
(43) Pub. Date: Apr. 10, 2014

(54) SYSTEM AND METHOD FOR PERFORMING A TRANSILIAC APPROACH FOR L5-S1 DISCECTOMY AND FUSION

(71) Applicant: Spineology, Inc., St. Paul, MN (US)

(72) Inventor: William D. Smith, Las Vegas, NV (US)

(73) Assignee: Spineology, Inc., St. Paul, MN (US)

(21) Appl. No.: 13/930,883

(22) Filed: Jun. 28, 2013

Related U.S. Application Data
(60) Provisional application No. 61/665,879, filed on Jun. 28, 2012.

(51) Int. Cl. A61F 2/44

(52) U.S. Cl. CPC A61F 2/4455
USPC 623/17.16

(57) ABSTRACT

A system, device and method for performing a transiliac approach for L5-S1 interbody fusion is disclosed. The surgeon may determine the access location using a neuromonitoring probe and fluoroscopy. Once the location is determined, the surgeon may use a cannulated needle or Jamshidi to create an initial pathway through the ilium. In an embodiment of the present invention, the surgeon may then advance a needle or Jamshidi through the ilium, just medial to the ilium wall. A guide pin may then be advanced through the cannulated needle or Jamshidi until the distal tip of the Jamshidi is reached. The Jamshidi may then be removed.

### PRIORITY

This application claims the benefit of U.S. Provisional Application Ser. No. 61/665,879, filed on Jun. 28, 2012, which is hereby incorporated herein by reference in its entirety.

### FIELD

The present invention relates generally to a system and method for performing a transiliac approach for L5-S1 interbody fusion.

### BACKGROUND

It is recognized that the spinal disc consists of three parts: first, the nucleus, a central portion that is a compression-resisting cushion; second, the annulus, a peripheral rim portion that is a tension-resisting hoop; and third, the end plates, the superior and inferior borders of the disc, consisting of the upper and lower surfaces of the vertebral body bones adjacent to the disc. Many studies have concluded that mechanical back pain is the most common and costly musculoskeletal condition affecting middle-aged humans in modern societies. Mechanical back pain may be caused by several factors, but overwhelming evidence suggests that degeneration of the spinal intervertebral disc, such as may be caused by Degenerative Disc Disease (DDD) is the most common condition causing back pain symptoms.

Like many other areas of surgery, spine surgery has become less invasive as smaller, more precise technology develops. Many minimally invasive intervertebral fusion devices exist, such as those disclosed in U.S. Pat. Nos. 5,571,189 and 5,549,679 and the commercially available XLIF® procedure by NuVasive. However, all minimally invasive fusion devices still require a surgical access opening that is as large as the device to be implanted. Generally speaking, the access aperture in minimally invasive procedures is at least 15-30 mm in diameter. Also, because minimally invasive procedures require direct visualization, the surgeon may need to cut bone and must significantly retract soft tissues and the nerve root, potentially causing nerve root injury or persistent post-operative pain.

Performing a L5-S1 fusion presents unique anatomical challenges. A lateral suprailiac approach into L5-S1 is often too steep relative to the axial plane of the disc space and therefore limits access for a complete discectomy and adequate endplate preparation for fusion.

Anterior and posterior approaches into L5-S1 require nerve root retraction which can lead to vascular injury. The retraction required in conventional approaches often leads to transient thigh pain and weakness of the hip flexor. Further, cutting through the iliac crest leads to site pain for the patient. The larger the fusion implant and instruments are, the more retraction is required and as such the higher the likelihood of vascular injury.

It is highly desired to provide an approach, instruments and an implant that will allow percutaneous transiliac access for fusion into L5-S1 without cutting the iliac crest and without retracting the nerve root. A percutaneous transiliac approach into L5-S1 yields an access to the disc space that is parallel to the L5-S1 endplates and that allows a thorough discectomy and better preparation of the fusion site.

The entire content of each and all patents, patent applications, articles and additional references, mentioned herein, are respectively incorporated herein in their entirety by reference.

The art described in this section is not intended to constitute an admission that any patent, publication or other information referred to herein is “prior art” with respect to this invention, unless specifically designated as such. In addition, this section should not be construed to mean that a search has been made or that no other pertinent information as defined in 37 C.F.R. §1.56(a) exists.

### SUMMARY

A system, device and method for performing a transiliac approach for L5-S1 interbody fusion is disclosed. The surgeon may determine the access location using a neuromonitoring probe and fluoroscopy. Once the location is determined, the surgeon may use a cannulated needle or Jamshidi to create an initial pathway through the ilium. In an embodiment of the present invention, the surgeon may then advance a needle or Jamshidi through the ilium, just medial to the ilium wall. A guide pin may then be advanced through the cannulated needle or Jamshidi until the distal tip of the Jamshidi is reached. The Jamshidi may then be removed.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts the guide pin exchange in the iliac crest according to various embodiments of the present invention.

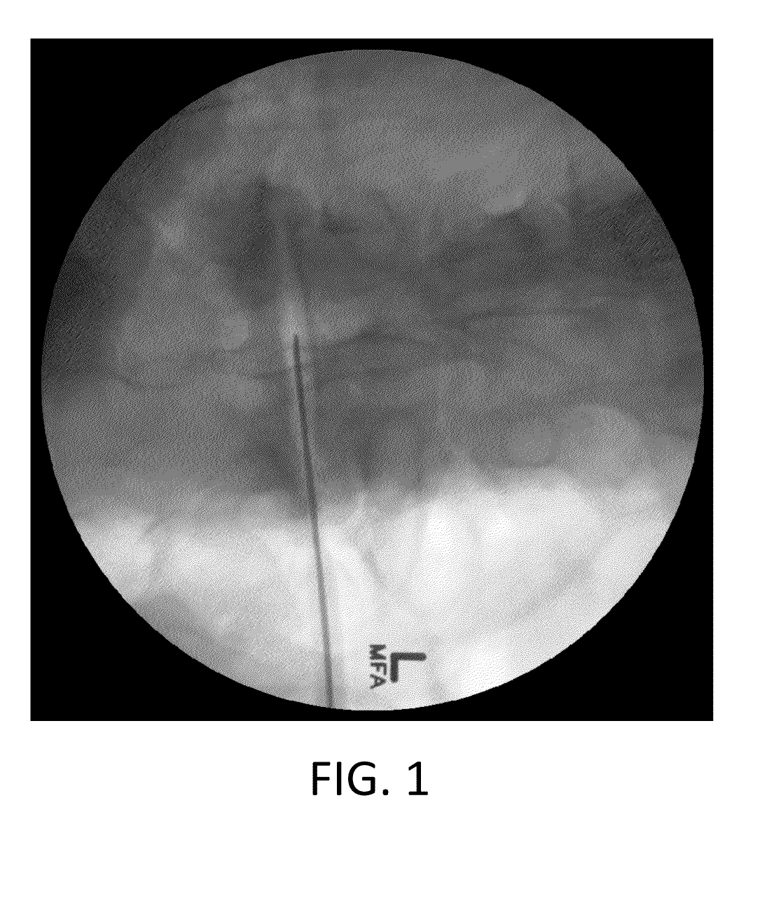

FIG. 2 depicts a first dilator advancing to the annulus according to various embodiments of the present invention.

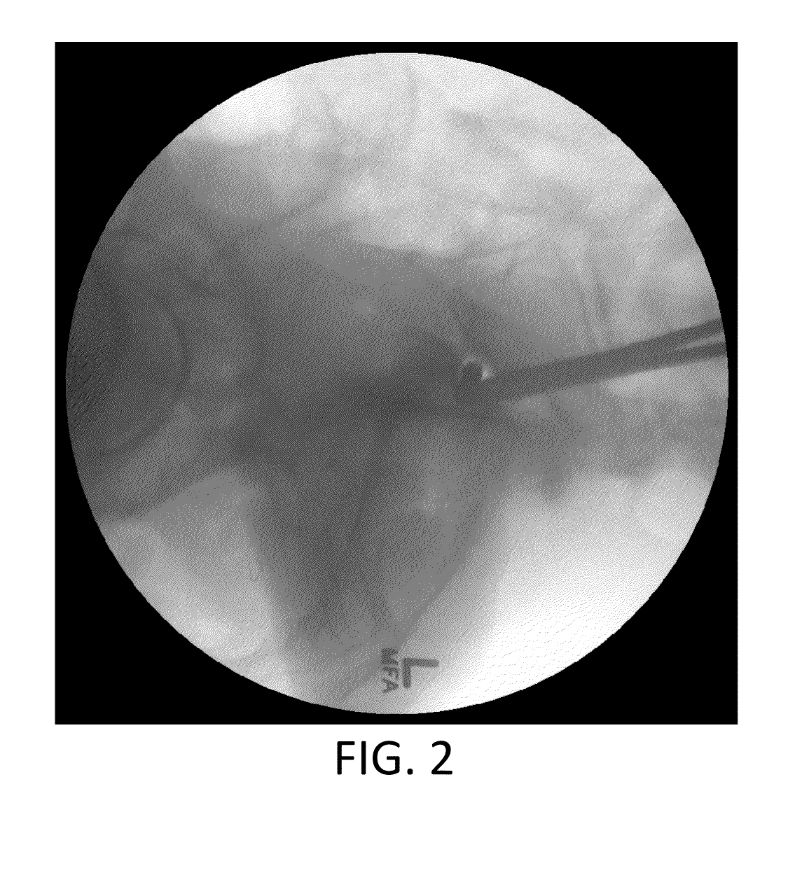

FIG. 3 depicts a second dilator according to various embodiments of the present invention.

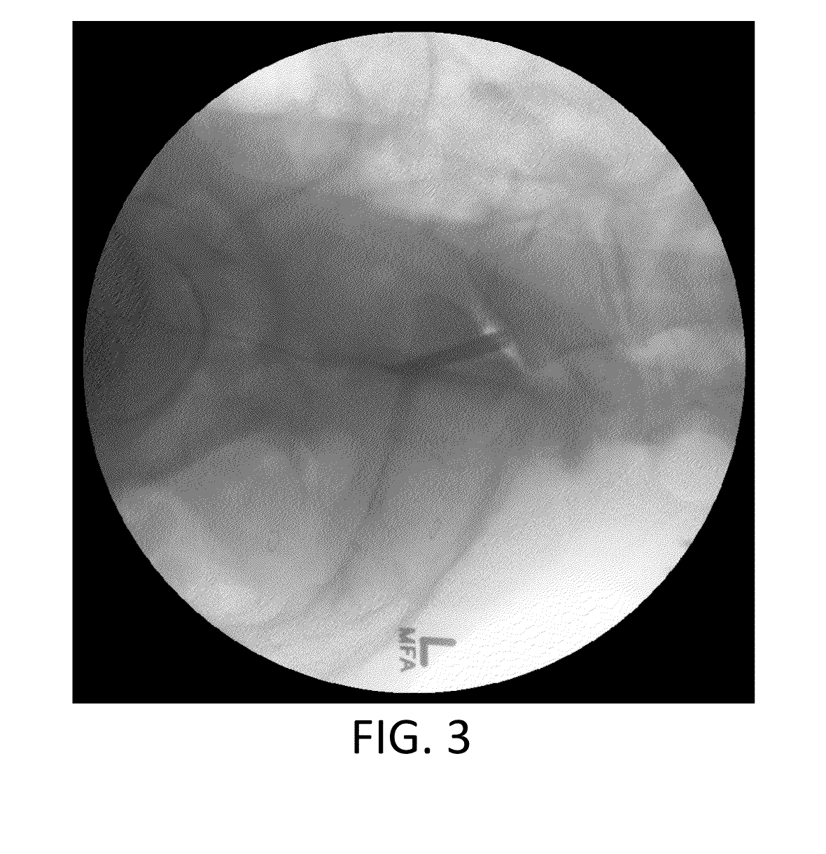

FIG. 4 depicts a working portal placed into the disc space according to various embodiments of the present invention.

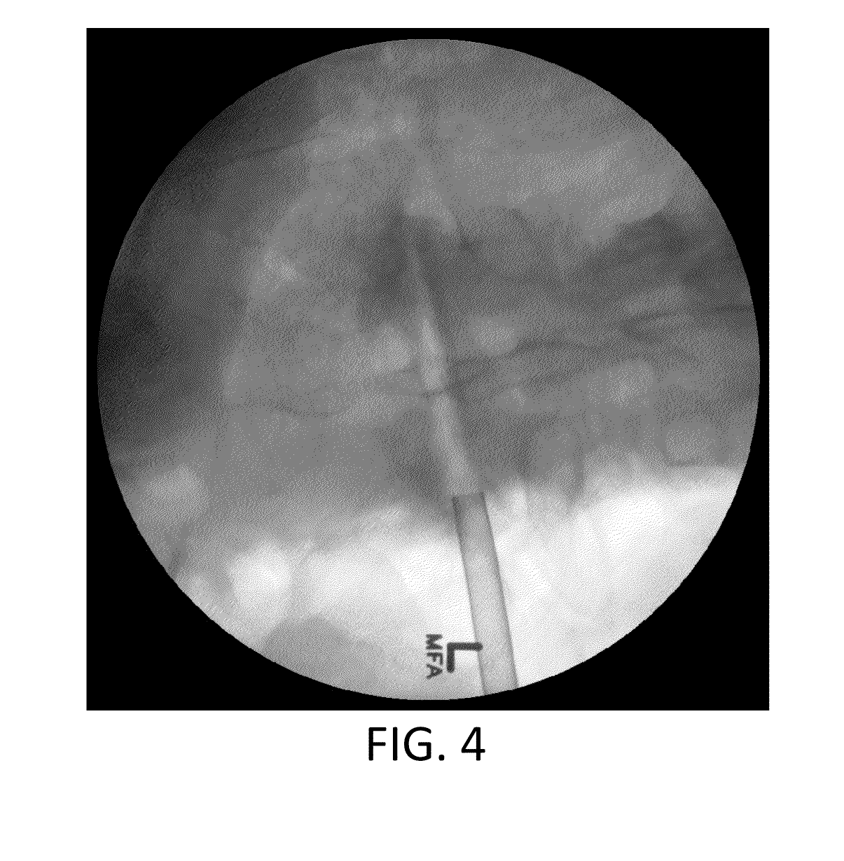

FIG. 5 depicts a drill, placed through the portal, used to evacuate nucleus material according to various embodiments of the present invention.

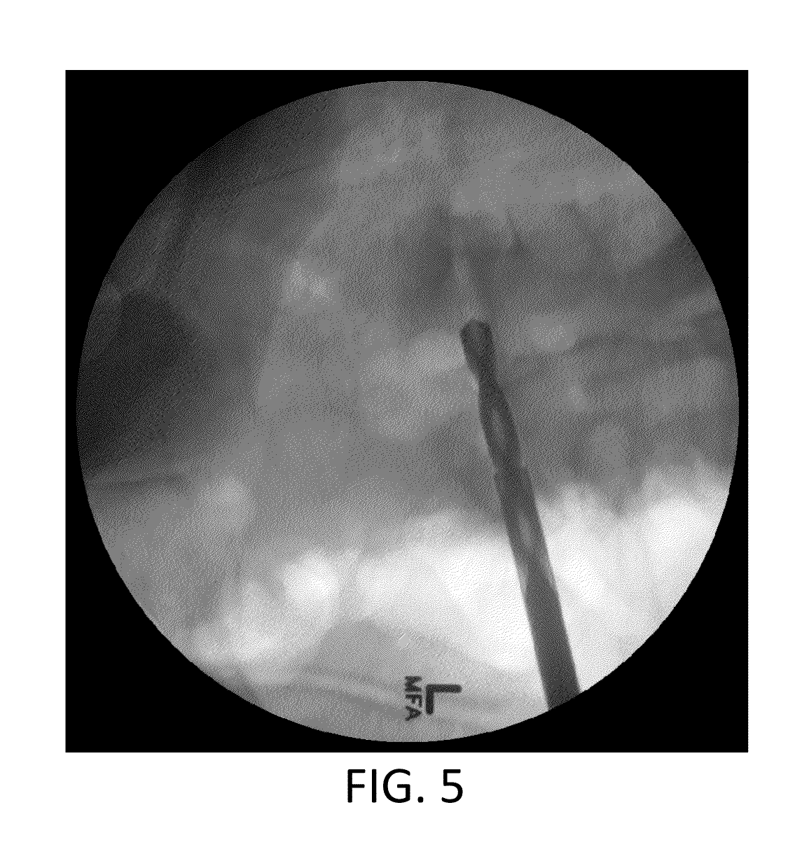

FIG. 6 depicts a brush for removing disc material according to various embodiments of the present invention.

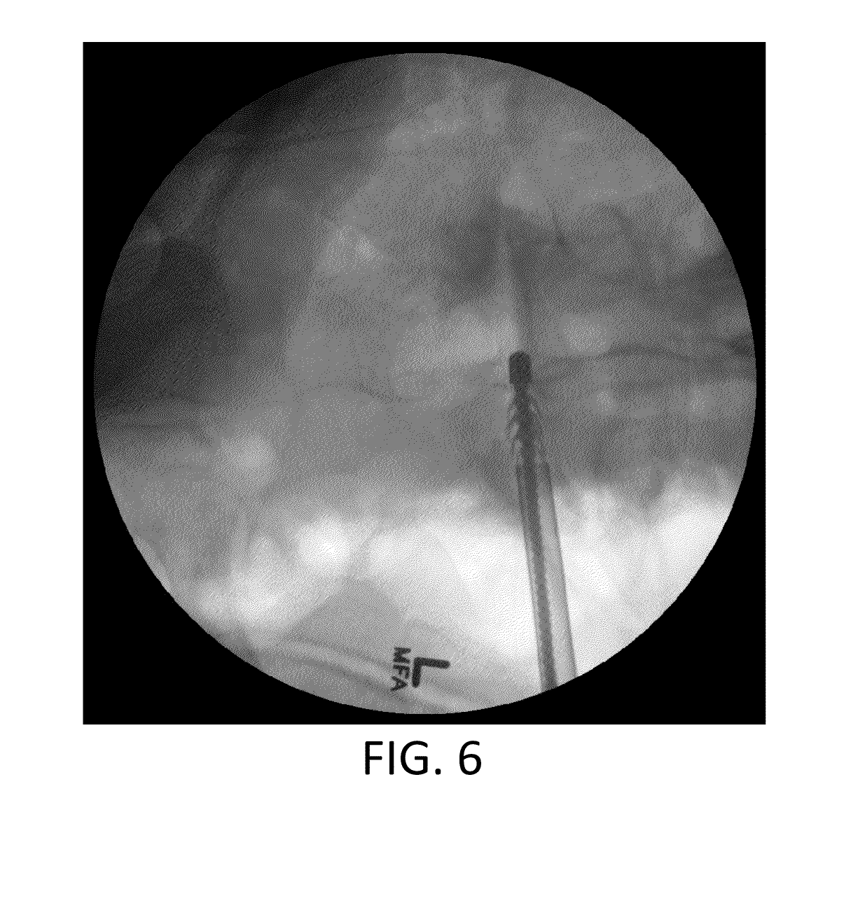

FIG. 7 depicts a shaper, placed through the portal, used to prepare the endplates according to various embodiments of the present invention.

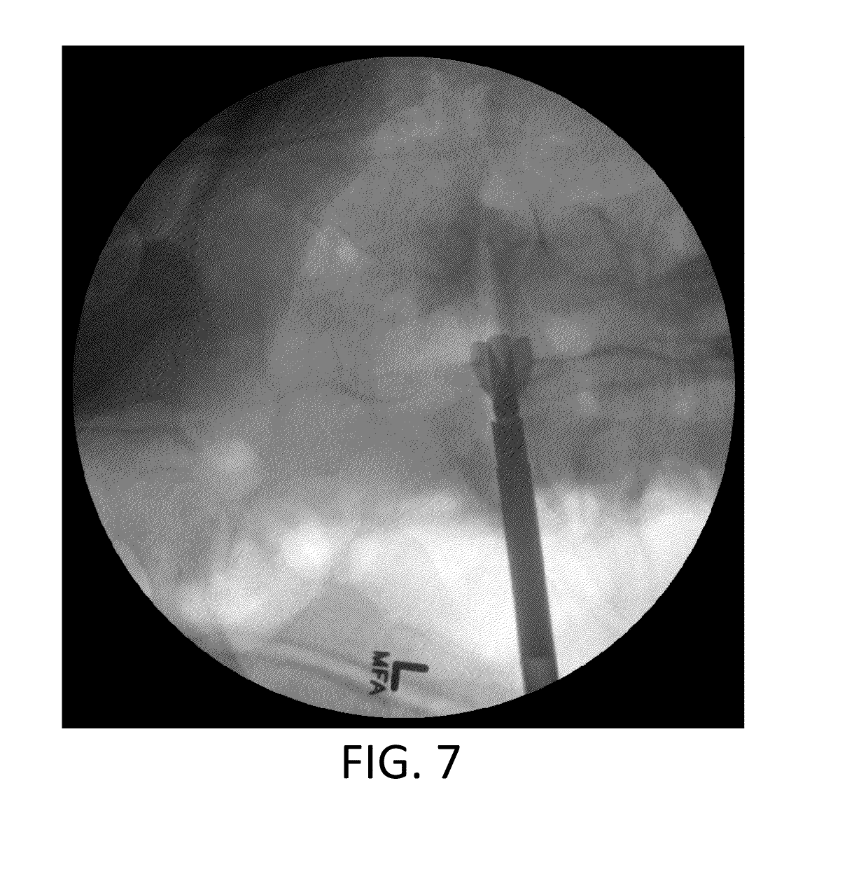

FIG. 8 depicts the percutaneous access opening according to various embodiments of the present invention.

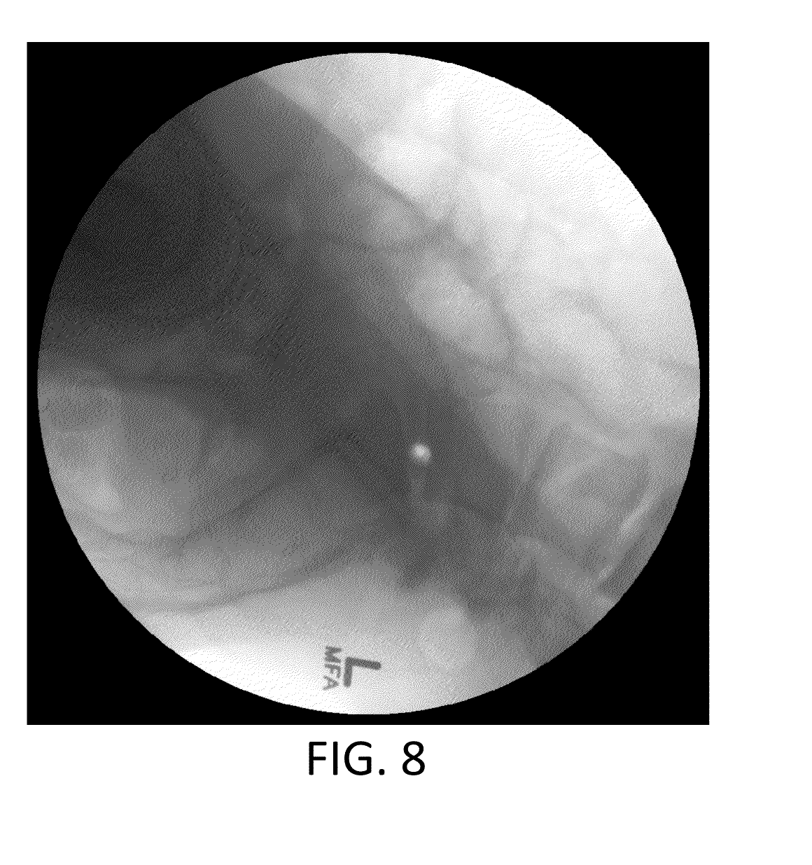

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

In an embodiment of the present invention the surgeon may determine the access location using a neuromonitoring probe and fluoroscopy. Once the location is determined, the surgeon may use a cannulated needle or Jamshidi to create an initial pathway through the ilium. In an embodiment of the present invention, the surgeon may then advance a needle or Jamshidi through the ilium, just medial to the ilium wall. A guide pin may then be advanced through the cannulated needle or Jamshidi until the distal tip of the Jamshidi is reached. The Jamshidi may then be removed.

According to one embodiment of the present invention, a first dilator may then be placed over the guide pin and through the ilium, just medial to the ilium wall. In one aspect of the present invention, the guide pin may now be removed, leaving the first dilator in place. A K-wire may now be placed through the first dilator. The first dilator may now be removed, leaving the K-wire in place. The surgeon may now sequentially dilate to create a channel in the ilium. A second dilator may then be placed over the guide pin and through the ilium. In one embodiment of the present invention, the guide pin may now be removed. According to one aspect of the present invention, a third dilator may now be placed over the second dilator. A fourth dilator may then be placed over the third dilator.

In one embodiment of the present invention, the dilators may not be advanced to the disc space. The dilators may be advanced through the ilium and just anterior to the ilium wall. According to one aspect of the present invention, the dilators sequentially create a channel in the ilium. This sequential dilation is in contrast to the standard cutting of the iliac crest and retraction of the nerve root.

According to one embodiment of the present invention, all of the dilators may now be removed. A neuromonitoring probe may then be placed through the created channel and advanced to the annulus. A guide pin may then be placed through the neuromonitoring probe and advanced into the disc space.

In one aspect of the present invention, a monitoring probe/dilator may be placed over the neuromonitoring probe. The neuromonitoring probe may then be removed leaving the guide pin in place with the monitoring probe/dilator. Next a dilator may be placed over the K-wire and through the monitoring probe/dilator. The dilator may then be advanced into the disc space.

In an embodiment of the present invention, the monitoring probe/dilator and guide pin may be removed leaving the dilator in place. A working portal, no greater than 8 mm in diameter may then be placed over the dilator and into the disc space. One the working portal is secured into place, the dilator may be removed.

According to one aspect of the present invention, a drill may be advanced into the disc space and advanced to the contralateral side to perform a release. In an embodiment of the present invention, the surgeon may now perform a percutaneous discectomy using live fluoroscopy to monitor progress. The discectomy may be accomplished using percutaneous instruments such as an articulating shaper as described in co-pending U.S. patent application Ser. No. 12/056,025 and brushes as described in co-pending U.S. Patent Application No. 61/506,021, both of which are hereby incorporated by reference herein in their entirety. The endplates may be scraped and prepared for fusion. The discectomy may be verified by using a device such as is described in co-pending U.S. patent application Ser. No. 12/650,889, which is hereby incorporated by reference herein in its entirety.

In an embodiment of the present invention, once the discectomy is accomplished the disc space is prepared for percutaneous placement of an interbody fusion device. Any fusion device that fits down a portal of no more than 8 mm in diameter may be used. In an embodiment of the present invention, a mesh container as described in U.S. Pat. No. 5,571,189 may be used as the fusion device, which is hereby incorporated by reference herein in its entirety.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A method for performing a percutaneous transiliac approach through the ilium into the L5-S1 intervertebral disc space for interbody fusion, comprising:
determining an access location using neuromonitoring;
placing a cannulated needle through the ilium to create an access path to the disc space through the ilium;
sequentially dilating to create a channel through the ilium;
placing a working portal having no greater than an 8 millimeter diameter into the disc space;
performing a percutaneous discectomy through the working portal; and
placing an interbody fusion device through the working portal.
